(19) United States
(12) Patent Application Publication
(10) Pub. No.: US 2015/0191412 A1
(43) Pub. Date: Jul. 9, 2015

(54) NANO-FILTRATION MEMBRANE AND METHOD OF PREPARING ORGANIC ACIDS USING THE SAME

(71) Applicants: Samsung Electronics Co., Ltd., Suwon-si (KR); IUCF-HYU (Industry-University Cooperation Foundation Hanyang University), Seoul (KR)

(72) Inventors: Jinkyu Kang, Daegu (KR); Jaeeun Kim, Hwaseong-si (KR); Changduk Kang, Gwacheon-si (KR); Ho bum Park, Seoul (KR); Younghoon Cho, Seoul (KR); Yoonsung Hwang, Seoul (KR); Kwangmyung Cho, Seongnam-si (KR)

(21) Appl. No.: 14/593,885

(22) Filed: Jan. 9, 2015

(30) Foreign Application Priority Data
Jan. 9, 2014 (KR) 10-2014-0003072

(51) Int. Cl. C07C 51/42; B01D 69/02; B01D 61/02; B01D 71/56; B01D 69/10

(52) U.S. Cl. CPC C07C 51/42; B01D 71/56; B01D 69/10; B01D 61/027; B01D 69/02; B01D 2325/00

(57) ABSTRACT

A nano-filtration membrane having a supporting membrane and a positively chargeable polymer layer adhered to at least one side of the supporting membrane, and method of using same to separate an organic acid from an organic acid-containing mixture.

### RELATED APPLICATIONS

This application claims the benefit of Korean Patent Application No. 10-2014-0003072, filed on Jan. 9, 2014, the entire disclosure of which is hereby incorporated by reference.

### BACKGROUND

1. Field

The present disclosure relates to a membrane, and more particularly, to a nano-filtration membrane. Further, the present disclosure relates to a method of preparing organic acids using the nano-filtration membrane.

2. Description of the Related Art

For instance, membranes may be classified into micro-filtration (MF) membranes, ultra-filtration membranes, nano-filtration membranes, and reverse osmosis membranes according to removal rating.

Nano-filtration membranes are generally known as membranes which allow permeation of monovalent ions and retention of divalent ions. Nano-filtration membranes generally have micro-pores having a pore size of about several nanometers, and are mainly used in retaining fine particles, molecules, ions, salts, etc in water (refer to US Patent Application Publication No. 2010/0190222).

Nano-filtration membranes are semipermeable or non-porous, and provide resolution based on molecular weights and ion charges. For instance, cutoff molecular weights of nano-filtration membranes for non-ionized molecules are usually placed between about 150 daltons and about 1,000 daltons. Molecules having molecular weights that are larger than the cutoff molecular weights may not permeate the nano-filtration membranes well, but molecules having molecular weights that are smaller than the cutoff molecular weights may permeate the nano-filtration membranes well. Among ions having the same molecular weight, the ions having larger ionic charges are often better retained by nano-filtration membranes while ions having smaller ionic charges often permeate the nano-filtration membranes better (refer to U.S. Pat. No. 5,503,750).

In general, pore sizes of surface layers of nano-filtration membranes are larger than those of reverse osmosis membranes. Further, the nano-filtration membranes may have negative charges in a wide pH range. Accordingly, nano-filtration membranes may effectively reject organic matters having a molecular weight of several hundreds of g/mol or more; polyvalent inorganic ions; heavy metals; etc. An attempt has been made to use such nano-filtration membranes in a separation process associated with a fermentation process. Examples of the separation process associated with the fermentation process may include recovery of solvents, concentration of fermented products, desalting of fermented liquids, etc.

In order to perform selective lactic acid separation for recovering lactic acids in the lactic acid fermentation process, (1) membranes having a high lactic acid rejection ratio and a high byproduct permeability or (2) membranes having a high lactic acid permeability and a high byproduct rejection ratio are required. Since sizes and molecular weights of lactic acids are usually smaller than those of byproducts, the membranes of type (2) would be more promising.

Reverse osmosis membranes have been known to exhibit a high lactic rejection ratio of from about 80% to about 99%. Therefore, the reverse osmosis membranes may be considered as the membranes of type (1). However, the reverse osmosis membranes not only reject lactic acids, but also strongly reject various organic matters and inorganic substances presented in fermented liquids. Therefore, the reverse osmosis membranes are not suitable for the selective lactic acid separation.

Nano-filtration membranes that are expected to exhibit high rejection ratios for byproducts having relatively large molecular weights may be considered as the membranes of type (2). Nano-filtration membranes that are commercially available are generally manufactured by interfacial polymerization. Such conventional nano-filtration membranes have carboxy groups in surfaces thereof. Accordingly, the conventional nano-filtration membranes may have negative charges. Since lactic acid also has a carboxy group, the lactic acid may also have negative charges. Accordingly, the conventional nano-filtration membranes strongly reject the lactic acid by repulsive electrical force. In general, the conventional nano-filtration membranes are known to exhibit lactic acid rejection ratios from about 50% to about 70%. Such lactic acid rejection ratios of the conventional nano-filtration membranes are so high that the conventional nano-filtration membranes cannot be used as the membranes of type (2).

Therefore, it is required to further lower the lactic acid rejection ratios of the nano-filtration membranes so as to effectively use the nano-filtration membranes as the membranes of type (2). In other words, nano-filtration membranes having higher lactic acid permeability are required.

Paragraphs [0030] to [0032] of US Patent Application Publication No. 2004/0033573 disclose: “Acidification includes controlling a pH to a value of lower than about 3.9, particularly a value of lower than about a pKa value (3.86) for lactic acid, usually a value of lower than about 3.8, preferably a value of lower than about 3.5, and more preferably a value from about 2.5 to about 3.0. As a result of that, the free lactic ions are bonded to hydrogen ions to form lactic acid, which does not have substantial electric charges accordingly. Therefore, free ions in a solution may include ions (that is, chlorine ions) derived from an inorganic acid used in acidification of a UF membrane permeate, ions derived from bases (that is, ammonia, NaOH or KOH) used in neutralization, and ions produced from small amounts of other incidentally existing salts. Thereafter, thus obtained acidic solution is usually treated in the nano-filtration process by using a nano-filtration membrane having a capacity of retaining divalent ions and molecules larger than about 180 g/mol. Monovalent ions are only partially retained, and small molecules that do not have electric charges freely permeate the nano-filtration membranes. Lactic acids that do not have electric charges in a low pH of the acidic solution permeate the nano-filtration membranes, and calcium and magnesium ions are retained along with relatively large molecules (that is, a residual sugar, a protein, and a coloring compound).”

For another instance, Paragraph [0037] of US Patent Application Publication No. 2010/0190222 discloses: “Preferably, a culture medium supplied to nano-filtration membranes in the step A has a pH from about 2.0 to about 4.5. Since, as they have been known, nano-filtration membranes reject or block ionized substances in a solution more easily than non-ionized substances in the solution, the culture medium is set at a pH of about 4.5 or less so that a ratio of lactic acids that are dissociated in the culture medium and existed in the form of lactic ions can be lowered, and thus, the lactic acids may easily permeate the nano-filtration membranes. If the pH is less than about 2.0, the nano-filtration membranes may be damaged. Moreover, the lactic acids have a pKa of about 3.86. Therefore, if the pH is set at about 3.86 or less, lactic acids that are not dissociated into lactic ions and hydrogen ions are more largely contained in the culture medium, and thus, the lactic acids may more effectively permeate the nano-filtration membranes, and this is more desirable. Additionally, controlling a pH of the culture medium may be performed during or after the microorganism fermentation process.”

Further, US Patent Application Publication No. 2010/0190222 reports that a permeability of lactic acids through nano-filtration membranes is from about 41.5% to about 57.8%. US Patent Application Publication No. 2010/0190222 defines the permeability of lactic acids as follows: permeability of lactic acid=the concentration of lactic acid in a solution which has permeated nano-filtration membranes÷(divided by) the concentration of lactic acid in a raw material solution supplied to the nano-filtration membranes.

Separation technology for separating lactic acids effectively and efficiently from a lactic acid-containing reaction mixture produced in the microorganism fermentation process may be required. The lactic acid has a molecular weight of 90. The lactic acid-containing reaction mixture includes residual saccharides (e.g., glucose) as well as lactic acid. Glucose has a molecular weight of 180. When considering the resolution capabilities of various previous separation processes, the molecular weights of the residual saccharides and those of the lactic acids are within molecular weight ranges that are similar to each other. Therefore, it is very difficult to separate the residual saccharides and lactic acids from each other.

### Summary

Provided are nano-filtration membranes having positively chargeable surfaces. Nano-filtration membranes of embodiments of the present disclosure may be used in separating organic acids effectively and efficiently from an organic acid-containing mixture produced in a microorganism fermentation process. Nano-filtration membranes of embodiments of the present disclosure may have high degrees of organic acid permeability and low degrees of saccharide permeability.

The disclosure provides, in one aspect, a nano-filtration membrane that includes: a supporting membrane; and a positively chargeable polymer layer adhered to at least one side of the supporting membrane, wherein the positively chargeable polymer layer has a surface zeta potential of about 5 mV or higher under a pH condition from about 2.0 to about 6.0.

According to another aspect of the present disclosure, an organic acid separating method is provided, which includes filtering an organic acid-containing nano-filtration feed through the nano-filtration membrane described herein to provide a nano-filtration permeate.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a scanning electron microscope photomicrograph of one surface of a polyacrylonitrile supporting membrane (UF grade, PAN 350, Sepromembranes, USA) used in Example 4;

FIG. 2 is a scanning electron microscope photomicrograph of the surface of an N6-polyamide layer formed on one surface of the polyacrylonitrile supporting membrane in Example 4; and

FIG. 3 is a scanning electron microscopic photomicrograph of the surface of a polyethyleneimine layer grafted on the surface of the N6-polyamid layer in Example 4.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

An embodiment of a nano-filtration membrane according to a first aspect of the present disclosure includes: a supporting membrane; and a positively chargeable polymer layer adhered to at least one side of the supporting membrane, wherein the positively chargeable polymer layer has a surface zeta potential of about 5 mV or higher under a pH condition from about 2.0 to about 6.0.

The supporting membrane may give mechanical strength to a nano-filtration membrane. Non-limiting examples of the supporting membrane may include nonwoven fabrics, micro-filtration (MF) membranes, ultra-filtration (UF) membranes, nano-filtration (NF) membranes, and laminates of two or more thereof. When the supporting membrane is an MF membrane or a UF membrane, nano-filtration resolution may be provided by the positively chargeable polymer layer. The supporting membrane may be a symmetrical membrane or an asymmetrical membrane. The asymmetrical membrane may have a dense layer in at least one side thereof. When the supporting membrane is an asymmetrical membrane, the positively chargeable polymer layer may be adhered to the surface of the dense layer of the asymmetrical membrane. The supporting membrane may be a single material membrane or a composite membrane. Non-limiting examples of forms of the supporting membrane may include a flat membrane form and a hollow fiber membrane form. When the supporting membrane has the form of a hollow fiber membrane, the positively chargeable polymer layer may be adhered to an outer surface and/or an inner surface of the hollow fiber membrane. Non-limiting examples of thickness values of the supporting membrane may include thickness values from about 50 micrometers to about 500 micrometers.

The supporting membrane may have any suitable pore size. For instance, an MF grade supporting membrane may have an average pore size or a nominal pore size in a range of from about 0.2 micrometers to about 10 micrometers. The MF grade supporting membrane may be manufactured from any suitable material, such as polyethylene, polypropylene, polyvinylidene fluoride, polytetrafluoroethylene, polysulfone, polyethersulfone, cellulose acetate, polyacrylonitrile, or any combinations thereof, but is not limited thereto.

The supporting membrane can have any suitable molecular weight cutoff. For instance, a UF grade supporting membrane may have a molecular weight cutoff from about 10,000 daltons to about 500,000 daltons. UF grade supporting membranes may be manufactured from any suitable material, such as polysulfone (PSF), polyethersulfone (PES), cellulose acetate (CA), polyacrylonitrile (PAN), or any combinations thereof, but is not limited thereto.

Also, an NF grade supporting membrane may have a molecular weight cutoff from about 200 daltons to about 1,000 daltons. NF grade supporting membranes may be manufactured from suitable materials, for instance, cellulose acetate, polyamide, polyester, polyimide, polyvinyl, or any combinations thereof, but is not limited thereto.

The positively chargeable polymer layer is adhered to at least one side of the supporting membrane. Adhesion of the positively chargeable polymer layer and the supporting membrane may include a physical adhesion and a chemical adhesion, but is not limited thereto. The physical adhesion may be realized by an ordinary LBL (layer-by-layer) type membrane manufacturing method. The chemical adhesion may be realized by chemical bonding between functional groups of the positively chargeable polymer layer and those of the supporting membrane.

Any positively chargeable polymer can be used. The surface of the positively chargeable polymer layer has a positive charge under nano-filtration membrane operating conditions. The positively chargeable polymer layer can have an impressive effect on the performance of the nano-filtration membrane. For instance, embodiments of a nano-filtration membrane of the present disclosure may exhibit excellent organic acid-permeating performance when the nano-filtration membrane is applied to the separation of organic acids from an organic acid-containing aqueous solution produced in the microorganism fermentation process. The organic acids in the organic acid-containing aqueous solution are presented in the form of “free” organic acids, i.e., organic acids that are not dissociated, or “organic acid ions”. The organic acid ions are anions. The positively chargeable polymer layer strongly attracts the anionic organic acid ions based on electrical attraction. Accordingly, the positively chargeable polymer layer strongly promotes permeation of the anionic organic acid ions through the positively chargeable polymer layer and the supporting membrane. In terms of a purpose of recovering an organic acid, the purpose accomplished by “permeating organic acid ions through a nano-filtration membrane” is the same as that accomplished by “permeating free organic acids through a nano-filtration membrane”. Organic acid permeation performance of a nano-filtration membrane of the present disclosure was substantially higher than free organic acid permeation using conventional nano-filtration membranes. As they have been known, the conventional nano-filtration membranes allow free organic acids to be permeated much better than organic acid ions. Nevertheless, free organic acid permeation of the conventional nano-filtration membranes is substantially lower than organic acid ion permeation of embodiments of a nano-filtration membrane of the present disclosure.

Residual saccharides are also contained in the organic acid-containing aqueous solution produced in the microorganism fermentation process. The saccharides are not dissociated in the organic acid-containing aqueous solution. Accordingly, the saccharides in the acid-containing aqueous solution are electrically neutral. The electrically neutral saccharides are not attracted by the positively chargeable polymer layer. Therefore, the nano-filtration membrane of the present disclosure does not promote permeation of saccharides. One of the important purposes of the organic acid separation process is to remove saccharides and recover organic acids. Embodiments of a nano-filtration membrane of the present disclosure may accomplish such a purpose of the organic acid separation process very effectively.

Positive-chargeability of the surface of the polymer layer may be indicated by a zeta potential. The zeta potential in the surface of the positively chargeable polymer layer is generated in a boundary between the surface and liquid. Such a boundary, which is called as a slip plane, is defined as a place where a stern layer and a diffusion layer meet. The stern layer is strongly bonded to the surface while the diffusion layer is not strongly bonded to the surface. An electrical potential in such a boundary may be used as an indicator of the positively-chargeability of the positively chargeable polymer layer. The zeta potential in the surface of the positively chargeable polymer layer may be easily measured under an electric field. Specifically, a zeta potential in the surface of a positively chargeable nano-filtration membrane may be measured by a zeta potential analyzer (ELSZ-1000, Otsuka electronics, Osaka, Japan). One method of measuring zeta potential is as follows: The positively chargeable nano-filtration membrane is immersed into a 10 mM NaCl aqueous solution. Mobility of NaCl electrolyte ions on the surface of the positively chargeable nano-filtration membrane is detected so that the zeta potential is measured. Measuring of the zeta potential is performed at two conditions of a pH 3 and a pH 7. Six measuring results are averaged with respect to each sample.

When it is defined in terms of its zeta potential, a positively chargeable polymer layer of the present disclosure is defined as a polymer layer having a positive zeta potential value at a pH of 6 or less.

For instance, a zeta potential in the surface of the positively chargeable polymer layer may be from about 5 mV to about 100 mV under pH conditions from about 2.0 to about 6.0. In another instance, the zeta potential in the surface of the positively chargeable polymer layer may be from about 5 mV to about 50 mV under pH conditions from about 3.9 to about 6.0.

Any polymers having the above-described range of zeta potential in the above-described range of pH may be used as the positively chargeable polymer.

For instance, the positively chargeable polymer layer may include polyamides (hereinafter referred to as “N₄₋₈ polyamides”) formed from an acyl chloride monomer and an amine monomer (hereinafter referred to as “N₄₋₈ amine monomers”) having 4 to 8 of —NH or —NH₂ groups. Further, the positively chargeable polymer layer may include polyamides formed from an acyl chloride monomer and an amine monomer having 5 to 7 of —NH or —NH₂ groups.

An N₄₋₈ polyamide layer has excellent positive-chargeability. For instance, the positively chargeable polymer layer comprising “N₄₋₈ polyamides” may have a surface zeta potential of about 5 mV or higher under a pH condition of 3.0.

The N₄₋₈ polyamide layer may additionally have an NF grade resolution. For instance, the N₄₋₈ polyamide layer may have a molecular weight cutoff from about 200 daltons to about 1,000 daltons.

The amine monomers mean monomers having —NH groups, —NH₂ groups, or —NH groups and —NH₂ groups. The N₄₋₈ amine monomers mean amine monomers having total 4 to 8 of —NH groups and —NH₂ groups. Examples of the N₄₋₈ amine monomers may include triethylenetetraamine (H(NHCH₂CH₂)₃NH₂), tetraethylenepentaamine (H(NHCH₂CH₂)₄NH₂), pentaethylenehexaamine (H(NHCH₂CH₂)₅NH₂), hexaethyleneheptaamine (H(NHCH₂CH₂)₆NH₂), heptaethyleneoctaamine (H(NHCH₂CH₂)₇NH₂), and any combinations thereof.

The acyl chloride monomers are monomers having —C(O)Cl groups. Representative examples of the acyl chloride monomers may include 1,3,5-benzentricarbonyl trichloride.

The N₄₋₈ polyamide layer may be formed by supplying an N₄₋₈ amine monomer aqueous solution to at least one side of the supporting membrane, and supplying an acyl chloride monomer organic solution to the one side of the supporting membrane to which the N₄₋₈ amine monomer aqueous solution has been supplied.

For instance, feeding the N₄₋₈ amine monomer aqueous solution to one or both sides of the supporting membrane may be performed by spraying, painting, pouring, dipping, etc.

An organic solvent that is not mixed with water may be used as an organic solvent used in the acyl chloride monomer organic solution. Examples of the organic solvent may include hexane, cyclohexane, heptane, C₈-C₁₂ alkanes, and any combinations thereof.

After feeding (e.g., supplying) the N₄₋₈ amine monomer aqueous solution to the surface of the supporting membrane, the acyl chloride monomer organic solution is fed (e.g., supplied) to the surface of the supporting membrane so that interfacial polymerization of N₄₋₈ amine monomers and acyl chloride monomers is initiated on the surface of the supporting membrane. A polyamide layer formed from the N₄₋₈ amine monomers and the acyl chloride monomers is formed on the surface of the supporting membrane.

Since the N₄₋₈ amine monomer aqueous solution and the acyl chloride monomer organic solution can penetrate into the supporting membrane, a portion of such formed N₄₋₈ polyamide layer may be infiltrated into and penetrate at least part of the supporting membrane. That is, the N₄₋₈ polyamide layer may root into the supporting membrane. Accordingly, an adhesive force between the N₄₋₈ polyamide layer and the supporting membrane may be quite strong. Due to such a strong adhesive force, the N₄₋₈ polyamide layer that is interfacially polymerized on the surface of the supporting membrane is difficult to separate from the supporting membrane. Therefore, nano-filtration membranes of such embodiments may maintain positive-chargeability for very long periods of time.

The molar ratio of the N₄₋₈ amine monomers to the acyl chloride monomers used in the interfacial polymerization may be controlled by a concentration of each solution, a feed amount of each solution, or a combination thereof. More specifically, the molar ratio of the N₄₋₈ amine monomers to the acyl chloride monomers may be, for example, from about 5:1 to about 10:1.

The thickness of the polyamide layer formed from the N₄₋₈ amine monomers and the acyl chloride monomers may be controlled by a concentration of each solution, a feed amount of each solution, a reaction time, or any combinations thereof. The thickness of the polyamide layer formed from the N4-8 amine monomers and the acyl chloride monomers may be, for example, in a range of about 100 nm to about 10 μm, or in a range of about 100 nm to about 1 μm.

The resolution (molecular weight cutoff) of the polyamide layer formed from the N₄₋₈ amine monomers and the acyl chloride monomers may be controlled by a concentration of each solution, a feed amount of each solution, or a combination thereof. The resolution (molecular weight cutoff) of the polyamide layer formed from the N4-8 amine monomers and the acyl chloride monomers may be, for example, in a range of about 100 Dalton to about 300 Dalton, or in a range of about 100 Dalton to about 200 Dalton.

The positive-chargeability of the N₄₋₈ polyamide layer formed from the N₄₋₈ amine monomers and the acyl chloride monomers may be controlled by a molar ratio of the N₄₋₈ amine monomers to the acyl chloride monomers, a concentration of each solution, a feed amount of each solution, or any combinations thereof.

The positively chargeable polymer layer may include, or be provided by, a cationic polymer electrolyte. Such a cationic polymer electrolyte layer has excellent positive-chargeability. For instance, the positively chargeable polymer layer comprising the cationic polymer electrolyte may have a surface zeta potential of about 5 mV or higher under a pH condition of 3.0.

If the cationic polymer electrolyte is used as a positively chargeable polymer, a filtration membrane having the NF grade resolution may be used as the supporting membrane when the cationic polymer electrolyte layer does not have an NF grade resolution.

Examples of the cationic polymer electrolyte may include polyethyleneimine (PEI), poly(allylamine hydrochloride) (PAH), poly(diallyldimethylammonium chloride) (PDADMAC), and any combinations thereof.

For instance, the formation of the cationic polymer electrolyte layer may be formed by applying a cationic polymer electrolyte solution to at least one side of the supporting membrane, and solidifying the applied cationic polymer electrolyte solution.

Examples of a solvent used in the cationic polymer electrolyte solution may include dimethyl sulfoxide (DMSO), hexadeuterodimethyl sulfoxide (DMSO-d6), ethylene glycol monomethyl ether (EGME), and any combinations thereof.

When a concentration of the cationic polymer electrolyte in the cationic polymer electrolyte solution is too low, the cationic polymer electrolyte layer formed on the supporting membrane may not have a surface zeta potential value of about 5 mV or higher under a pH condition of 3.

When the concentration of the cationic polymer electrolyte in the cationic polymer electrolyte solution is too high, the cationic polymer electrolyte layer formed on the supporting membrane becomes so dense that water permeability, organic acid permeability, etc may be lowered. The concentration of the cationic polymer electrolyte in the cationic polymer electrolyte solution may be, for example, in a range of about 0.01 wt % to about 5 wt %, or in a range of about 0.05 wt % to about 1 wt %.

The cationic polymer electrolyte solution may be applied to at least one side of the supporting membrane by any suitable method, such as by spraying, painting, pouring, dipping, etc.

Solidifying the applied cationic polymer electrolyte solution may be performed, for instance, by removing a solvent in the cationic polymer electrolyte solution, such as by volatilization, evaporation, washing, etc.

While the cationic polymer electrolyte solution applied to the side of the supporting membrane is being solidified or after the cationic polymer electrolyte solution applied to the side of the supporting membrane is solidified, chemical bonding may occur between functional groups of the cationic polymer electrolyte and those of the supporting membrane. Accordingly, the cationic polymer electrolyte layer and the supporting membrane may be grafted together. An adhesion force between the cationic polymer electrolyte layer and the supporting membrane may be quite strong by such a grafting reaction. Due to such an excellent adhesive force, the cationic polymer electrolyte layer that is grafted on the surface of the supporting membrane may be very hard to separate from the supporting membrane. Therefore, nano-filtration membranes of such embodiments may maintain positive-chargeability for very long periods of time.

For instance, the grafting reaction may occur between the amine group of the cationic polymer electrolyte and an non-reacted acyl chloride group of the supporting membrane when the cationic polymer electrolyte has amine groups, i.e., —NH groups or —NH2 groups, and the supporting membrane includes polyamides formed from amine monomers and acyl chloride monomers. Examples of the cationic polymer electrolyte having an amine group may include polyethyleneimine (PEI), poly(allylamine hydrochloride) (PAH), and any combinations thereof.

The membrane can comprise both a positively chargeable polymer layer and a cationic polymer electrolyte layer. In one embodiment, the positively chargeable polymer layer may include: an N₄₋₈ polyamide layer formed from N₄₋₈ amine monomers and acyl chloride monomers on the supporting membrane; and a cationic polymer electrolyte layer formed on the N₄₋₈ polyamide layer. The positively chargeable polymer layer may include: an N₅₋₇ polyamide layer formed from N₅₋₇ amine monomers and acyl chloride monomers on the supporting membrane; and a cationic polymer electrolyte layer formed on the N₅₋₇ polyamide layer. In this case, the positively chargeable polymer layer including the N₄₋₈ polyamide layer and the cationic polymer electrolyte layer may exhibit excellent positive-chargeability since positive-chargeability of the N₄₋₈ polyamide layer and that of the cationic polymer electrolyte layer function compositely.

In another embodiment, the positively chargeable polymer layer may include: an N₄₋₈ polyamide layer formed from N₄₋₈ amine monomers and acyl chloride monomers on the supporting membrane; and a cationic polymer electrolyte layer having amine groups formed on the N₄₋₈ polyamide layer. The positively chargeable polymer layer may include: an N₅₋₇ polyamide layer formed from N₅₋₇ amine monomers and acyl chloride monomers on the supporting membrane; and a cationic polymer electrolyte layer having amine groups formed on the N₅₋₇ polyamide layer. In this case, the positively chargeable polymer layer including the N₄₋₈ polyamide layer and the cationic polymer electrolyte layer may exhibit increased positive-chargeability since positive-chargeability of the N₄₋₈ polyamide layer and that of the cationic polymer electrolyte layer may function compositely. Further, the cationic polymer electrolyte layer and the N₄₋₈ polyamide layer are grafted through chemical bonding between the amine group of the cationic polymer electrolyte layer and a non-reacted acyl chloride group of the N₄₋₈ polyamide layer. Accordingly, adhesive force between the cationic polymer electrolyte layer and the N₄₋₈ polyamide layer is reinforced. Moreover, the N₄₋₈ polyamide layer formed by interfacial polymerization on the supporting membrane may be rooted into the supporting membrane. Therefore, adhesive force between the N₄₋₈ polyamide layer and the supporting membrane is also reinforced. As a result, the positively chargeable polymer layer may include the cationic polymer electrolyte layer having an amine group and the N₄₋₈ polyamide layer is strongly adhered to the supporting membrane. Therefore, nano-filtration membranes of such embodiments may exhibit much reinforced positive-chargeability for very long periods of time.

Hereinafter, a method of separating an organic acid according to a second aspect of the present disclosure is described.

An embodiment of a method of separating an organic acid according to a second aspect of the present disclosure includes filtering a nano-filtration feed containing an organic acid through embodiments of a nano-filtration membrane according to a first aspect of the present disclosure to provide a nano-filtration permeate.

Ordinary examples of the nano-filtration feed containing an organic acid may include a culture solution produced in the organic acid fermentation process using microorganisms.

Examples of the microorganisms used in the organic acid fermentation process may include: yeasts such as baker's yeast (e.g., Saccharomyces cerevisiae); bacteria such as Bacillus coli (e.g., E. coli) and Coryneform bacteria; filamentous fungi; actinomycetes; culture cells such as animal cells and insect cells; etc.

Any materials which promote the growth of microorganisms to produce organic acids may be used as nutrients used in the organic acid fermentation process. Examples of the nutrients may include carbon sources, nitrogen sources, inorganic salts, and organic trace nutrients such as amino acid, vitamin, etc.

Examples of the carbon sources may include: sugars such as glucose, sucrose, fructose, galactose and lactose; a saccharified starch solution containing the sugars; molasses such as sweet potato molasses, beet molasses and high-test molasses; an organic acid like an acetic acid; an alcohol like ethanol; glycerin; etc.

Examples of the nitrogen sources may include an ammonia gas, an ammonia aqueous solution, an ammonium salt, a urea, a nitrate, an oilcake, a soybean hydrolysate, a casein digest, other amino acids, a vitamin, a corn steep liquor, a yeast or yeast extract, a meat extract, a peptide (e.g., peptone), etc.

Examples of the inorganic salts may include phosphates, magnesium salts, calcium salts, iron salts, manganese salts, etc.

Conditions for the organic acid fermentation process may include a pH from about 4 to about 8, and a temperature from about 20° C. to about 40° C.

In one embodiment, the nano-filtration feed containing an organic acid may be a UF permeate obtained by filtering a culture solution produced in the organic acid fermentation process using microorganisms through an UF membrane to provide the ultra-filtration (UF) permeate.

Examples of the UF membrane may include UF membranes having a molecular weight cutoff from about 10,000 daltons to about 500,000 daltons.

For instance, particulate matters, molecules having a high molecular weight of 10,000 or higher, etc in the culture solution may be removed through the ultra-filtration process.

Using the UF permeate of the culture solution as the nano-filtration feed containing an organic acid may prevent a nano-filtration membrane from being contaminated or fouled by particulate matters and molecules of a high molecular weight.

Examples of the organic acids in the nano-filtration feed containing an organic acid may include formic acid, acetic acid, lactic acid, succinic acid, butyric acid, propionic acid, valeric acid, isovaleric acid, capronic acid, heptanoic acid, octanoic acid, oxalic acid, maloic acid, glutaric acid, adipic acid, glycolic acid, glycinic acid, acrylic acid, tartaric acid, fumaric acid, benzoic acid, maleric acid, phthalic acid, and salicylic acid.

The organic acids exist in the form of organic acid ions or free organic acids in the nano-filtration feed containing an organic acid. When the nano-filtration feed containing an organic acid has a too low pH value, equilibrium moves toward the free organic acids. Accordingly, the fraction of the organic acid ions in the nano-filtration feed containing an organic acid may be extremely lowered, and thus, the amount of organic acid ions to be attracted by the positively chargeable nano-filtration membrane may be very little. As a pH value of the nano-filtration feed containing an organic acid increases, equilibrium moves toward the organic acid ions. Accordingly, the fraction of the organic acid ions in the nano-filtration feed containing an organic acid are increased. Therefore, the positively chargeable nano-filtration membrane may attract a large amount of the organic acid ions.

For instance, the nano-filtration feed containing an organic acid may have a pH value from about 2 to about 6. For another instance, the nano-filtration feed containing an organic acid may have a pH value from about 3.9 to about 6.0. For another instance, the nano-filtration feed containing an organic acid may have a pH value from about 4.5 to about 6.0.

In another instance, the nano-filtration feed containing an organic acid may have a pH value higher than a pKa value of the organic acid. For instance, when the organic acid is lactic acid having a pKa value of 3.86, a nano-filtration feed containing lactic acid may have a pH value of higher than 3.86 or not lower than 3.9.

In order for the nano-filtration feed containing an organic acid to have a desired pH range, the nano-filtration feed containing an organic acid may additionally include a pH adjuster.

Water, organic acid ions and other components capable of permeating a nano-filtration membrane permeate the positively chargeable nano-filtration membrane, thus providing a nano-filtration permeate. Since the positively chargeable nano-filtration membrane attracts organic acid ions very strongly, an organic acid permeability through the nano-filtration membrane is very high. The organic acid permeability through the nano-filtration membrane is defined as “the concentration of an organic acid in the nano-filtration permeate (including free organic acid and organic acid ions)÷(divided by) the concentration of an organic acid in the nano-filtration feed (including a free organic acid and organic acid ions).”

For instance, the nano-filtration permeate may have a high organic acid concentration ensuring that the organic acid permeability through the nano-filtration membrane becomes about 70% or higher (e.g., about 75% or higher). For another instance, the nano-filtration permeate may have a high organic acid concentration ensuring that the organic acid permeability through the nano-filtration membrane becomes about 80% or higher.

The positively chargeable nano-filtration membrane does not strongly attract saccharides. Therefore, a saccharide permeability through the nano-filtration membrane is low. The saccharide permeability through the nano-filtration membrane is defined as “saccharide concentration of the nano-filtration permeate÷(divided by) saccharide concentration of the nano-filtration feed.”

For instance, the nano-filtration permeate may have a low saccharide concentration ensuring that the saccharide permeability through the nano-filtration membrane becomes about 35% or lower. For another instance, the nano-filtration permeate may have a low saccharide concentration ensuring that the saccharide permeability through the nano-filtration membrane becomes about 25% or lower.

The nano-filtration permeate may be directly used as an organic acid product. Further, after the nano-filtration permeate is treated in one or more additional subsequent processes, the treated nano-filtration permeate may become an organic acid product. Examples of subsequent processes may be ion exchange processes, concentration processes, or a combination thereof. Accordingly, other embodiments of the organic acid separating method may further include: treating the nano-filtration permeate with an ion exchange resin; concentrating the nano-filtration permeate; distilling the nano-filtration permeate; or any combinations thereof.

For instance, concentrating the nano-filtration permeate may include heating the nano-filtration permeate to evaporate water in the nano-filtration permeate.

For instance, distilling the nano-filtration permeate may include heating the nano-filtration permeate to evaporate an organic acid in the nano-filtration permeate and recover the evaporated organic acid.

Hereinafter, the present disclosure is described more in detail through the following Examples. However, the present disclosure is not particularly limited to the following Examples.

### EXAMPLES

### Summary

A polymer electrolyte was grafted on the surface of a nano-filtration membrane manufactured by interfacial polymerization so that a selection layer was formed on the surface of the nano-filtration membrane. Grafting was performed through chemical bonding between a non-reacted acyl chloride group remained in the nano-filtration membrane and an amine group presenting in the polymer electrolyte.

First, rejection ratios for lactic acid and glucose of a nano-filtration membrane formed on the surface of a ultra-filtration (UF) membrane through interfacial polymerization were measured.

Next, a polymer electrolyte was grafted on the surface of nano-filtration membranes manufactured according to the present disclosure. Nano-filtration membranes of the present disclosure on surfaces of which the polymer electrolyte was grafted had lower water permeabilities than nano-filtration membranes on surfaces of which the polymer electrolyte was not grafted. However, nano-filtration membranes of the present disclosure on surfaces of which the polymer electrolyte was grafted had lower lactic acid rejection ratios and higher glucose rejection ratios than nano-filtration membranes of the present disclosure on surfaces of which the polymer electrolyte was not grafted.

Measurement of Zeta Potentials

The surface zeta potentials of positively chargeable nano-filtration membranes were measured using a zeta potential analyzer (ELSZ-1000, Otsuka electronics, Osaka, Japan). The positively chargeable nano-filtration membranes were immersed into a 10 mM NaCl aqueous solution. The mobility of NaCl electrolyte ions in the surfaces of the positively chargeable nano-filtration membranes was detected to measure the zeta potentials. The measurement of the zeta potentials was conducted at two conditions of a pH 3 and a pH 7. Six measurement results were averaged for each of the samples.

Measurement of Lactic Acid Rejection Ratios and Glucose Rejection Ratios

First, 1 g of lactic acid (L-(+)-Lactic acid, Sigma Aldrich, St. Louis, Mo., USA) and 1 g of glucose (D-(+)-Glucose, Sigma Aldrich, St. Louis, Mo., USA) were dissolved into 1,000 g of ultrapure water to prepare a nano-filtration feed. The nano-filtration feed had a temperature of 25° C., a pH value of 3.0, a lactic acid concentration of 1 g/L and a glucose concentration of 1 g/L. Next, a nano-filtration permeate was obtained by supplying the nano-filtration feed to a flat film type membrane separation device on which one of nano-filtration membranes of Examples 1 to 5 and Comparative Examples 1 and 2 was mounted. An effective area of the nano-filtration membrane was 14.6 cm², and a pressure applied to the nano-filtration membrane was 200 psi. Thereafter, the amount of the nano-filtration permeate, and lactic acid and glucose concentrations of the nano-filtration permeate were measured. A lactic acid rejection ratio is defined as “[1-(lactic acid concentration of the nano-filtration permeate/lactic acid concentration of the nano-filtration feed)]×100%.” A glucose rejection ratio is defined as “[1-(glucose concentration of the nano-filtration permeate/glucose concentration of the nano-filtration feed)]×100%.”
pH measuring instrument: pH meter (Orion 5-star, Thermo Fisher Scientific, Waltham, Mass., USA)
Lactic acid concentration-measuring instrument: total organic carbon analyzer (Multi N/C 3100, Analytikjena, Jena, Germany)
Glucose concentration-measuring instrument: total organic carbon analyzer (Multi N/C 3100, Analytikjena, Jena, Germany)

### Example 1

### UF Grade PSF Supporting Membrane/N5-Polyamide Layer

In Example 1, a nano-filtration membrane having a UF grade polysulfone (PSF) film as a supporting membrane and having an N5-polyamide layer as a nano-filtration membrane was prepared. First, 3.39 g of tetraethylenepentaamine (H(NHCH₂CH₂)₄NH₂) was dissolved into 98.5 g of deionized water to obtain a tetraethylenepentaamine aqueous solution. Further, 0.5 g of 1,3,5-benzenetricarbonyltrichloride (Sigma Aldrich, USA) was dissolved into an liter of an organic solvent (ISOPAR) to obtain a 1,3,5-benzenetricarbonyl trichloride organic solution. Next, a PSF supporting membrane was dipped into a tetraethylenepentaamine aqueous solution for 3.5 minutes. After removal of the membrane, the tetraethylenepentaamine aqueous solution remained on the surface of the PSF supporting membrane, and the PSF supporting membrane was then dipped into the 1,3,5-benzenetricarbonyl trichloride organic solution for one minute. Then, the PSF supporting membrane was washed with n-hexane to obtain a flat film type nano-filtration membrane of Example 1. The prepared nano-filtration membrane of Example 1 was immersed and stored in ultrapure water.

### Example 2

### UF Grade PSF Supporting Membrane/N6-Polyamide Layer

A nano-filtration membrane of Example 2 was prepared in the same method as Example 1 except that pentaethylenehexaamine (H(NHCH₂CH₂)₅NH₂) instead of tetraethylenepentaamine (H(NHCH₂CH₂)₄NH₂) was used.

### Comparative Example 1

### UF Grade PSF Supporting Membrane/N3-Polyamide Layer

A nano-filtration membrane of Comparative Example 1 was prepared in the same method as Example 1 except that diethylenetriamine (H(NHCH₂CH₂)₂NH₂) instead of tetraethylenepentaamine (H(NHCH₂CH₂)₄NH₂) was used.

<Analysis of Permeating Characteristics According to Types of Amine Monomers>

Permeating characteristics of nano-filtration membranes of Examples 1 and 2 and Comparative Example 1 produced by interfacial polymerizing different types of amine monomers were analyzed. Analysis results were summarized in Table 1.

TABLE 1

| | Water per- | Lactic acid | Glucose |
|---|---|---|---|
| | meability | rejection | rejection |
| Sample | (LMH/bar) | ratio (%) | ratio (%) |
| Comparative | 3.6 | 80 | 79 |
| Example 1 (PSF-N3) | | | |
| Example 1 (PSF-N5) | 4.5 | 56 | 80 |
| Example 2 (PSF-N6) | 7 | 29 | 67 |

As represented in Table 1, water permeability values were improved according as the number of —NH groups was increased. Further, the lactic acid rejection ratios were decreased according as the number of —NH groups was increased. On the other hand, according as the number of —NH groups was increased, the glucose rejection ratios were not decreased or much less decreased compared to the lactic acid rejection ratio. Therefore, it can be seen that nano-filtration membranes which selectively permeate lactic acid better than glucose may be obtained by increasing the number of —NH groups.

### Example 3

### UF Grade PAN Supporting Membrane/N5-Polyamide Layer

In Example 3, a nano-filtration membrane of Example 3 was prepared in the same method as Example 1 except that PAN 350 (Sepromembranes, Oceanside, Calif., USA), a UF grade polyacrylonitrile (PAN) membrane, instead of PSF was used as a supporting membrane.

### Comparative Example 2

### UF Grade PAN Supporting Membrane/N3-Polyamide Layer

A nano-filtration membrane of Comparative Example 2 was prepared in the same method as Example 3 except that diethylenetriamine (H(NHCH₂CH₂)₂NH₂) instead of tetraethylenepentaamine (H(NHCH₂CH₂)₄NH₂) was used.

### Example 4

### UF Grade PAN Supporting Membrane/N6-Polyamide Layer/0.1 wt % PEI Layer

In Example 4, a positively chargeable nano-filtration membrane using a UF grade polyacrylonitrile (PAN) membrane as a supporting membrane and using an N6-polyamide layer and a polyethyleneimine (PEI) layer was prepared. The formation of the PEI layer was conducted by using a PEI solution having 0.1% by weight of a PEI concentration.

The formation of an N6-polyamide layer: First, 3.39 g of pentaethylenehexaamine (H(NHCH₂CH₂)₅NH₂: Sigma Aldrich, USA) was dissolved into 98.5 g of deionized water to obtain a pentaethylenehexaamine aqueous solution. Further, 0.5 g of 1,3,5-benzenetricarbonyltrichloride (Sigma Aldrich, USA) was dissolved into 1 liter of an organic solvent ISOPAR to obtain a 1,3,5-benzenetricarbonyltrichloride organic solution. Next, a surface of a PAN supporting membrane (UF grade, PAN 350, Sepromembranes, USA) was impregnated with a pentaethylenehexaamine aqueous solution for 3.5 minutes. Next, after removing a pentaethylenehexaamine aqueous solution remained on the surface of the PAN supporting membrane, the surface of the PAN supporting membrane was impregnated with the 1,3,5-benzenetricarbonyltrichloride organic solution for one minute. Thereafter, the PAN supporting membrane was washed with n-hexane to obtain a PAN supporting membrane with the N6-polyamide layer. This membrane was immersed and kept in ultrapure water.

The formation of a 0.1 wt % PEI layer: Thereafter, 0.1 g of polyethyleneimine (PEI) (Sigma Aldrich, USA) was dissolved into 100 g of ultrapure water to obtain a 0.1 wt % PEI solution. Next, the PAN supporting membrane with the N6-polyamide layer was dipped into the 0.1 wt % PEI solution for 3 minutes. Accordingly, a positively chargeable nano-filtration membrane of Example 4 in which an N6-polyamide layer and a polyethyleneimine (PEI) layer were sequentially laminated on one surface of the PAN supporting membrane was obtained. The positively chargeable nano-filtration membrane of Example 4 was dipped and kept in ultrapure water.

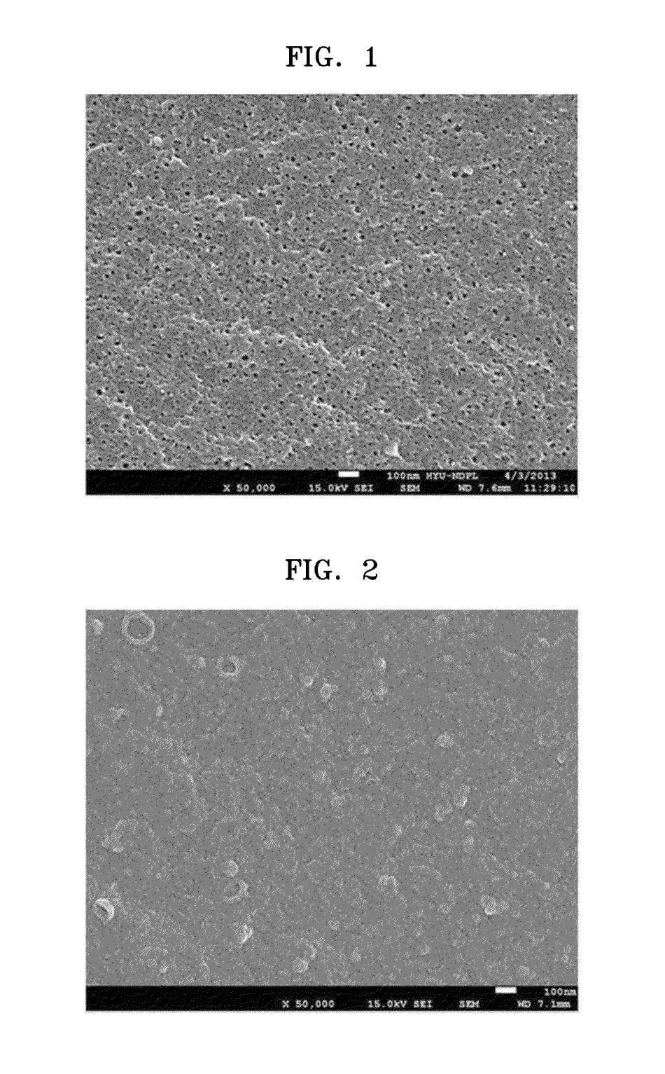

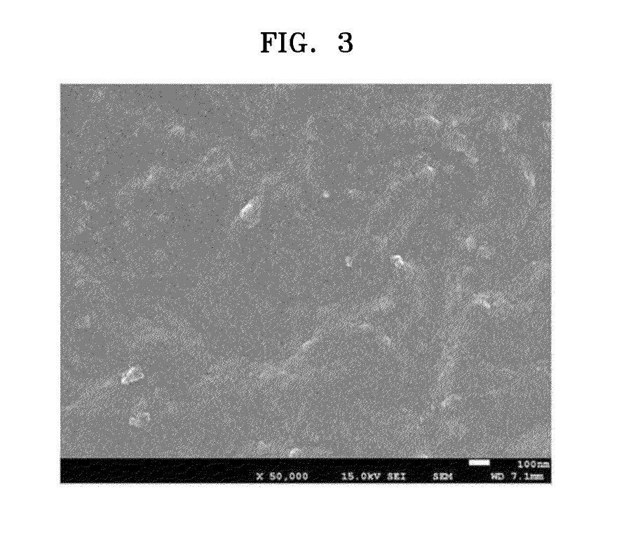

FIG. 1 is a scanning electron microscopic photograph illustrating one surface of a polyacrylonitrile supporting membrane (UF grade, PAN 350, Sepromembranes, USA) used in Example 4. FIG. 2 is a scanning electron microscopic photograph illustrating the surface of an N6-polyamid layer formed on one surface of the polyacrylonitrile supporting membrane in Example 4. FIG. 3 is a scanning electron microscopic photograph illustrating the surface of a polyethyleneimine layer grafted on the surface of the N6-polyamid layer in Example 4.

### Example 5

### UF Grade PAN Supporting Membrane/N6-Polyamide Layer/1.0 wt % PEI Layer

In Example 5, a positively chargeable nano-filtration membrane was prepared in the same method as Example 4 except that the PEI solution had 1.0% by weight of the PEI concentration.

### Example 6

### UF Grade PAN Supporting Membrane/N3-Polyamide Layer/1.0 wt % PEI Layer

In Example 6, a positively chargeable nano-filtration membrane was prepared in the same method as Example 5 except that diethylenetriamine (H(NHCH₂CH₂)₂NH₂) instead of pentaethylenehexaamine (H(NHCH₂CH₂)₅NH₂) was used.

<Analysis of Zeta Potentials and Permeation Characteristics of Nano-Filtration Membranes>

Analysis results of zeta potentials and permeation characteristics for nano-filtration membranes of Examples 1 to 6 and Comparative Examples 1 and 2 are presented in Table 2.

TABLE 2

| | Water per- | Lactic acid | Glucose | Surface zeta | |
|---|---|---|---|---|---|
| | meability | rejection | rejection | potential (mV) | |
| Samples | (LMH/bar) | ratio (%) | ratio (%) | pH 3 | pH 7 |
| Comparative | 3.6 | 80 | 79 | 2.8 | −37.0 |
| Example 1 | | | | | |
| (PSF/N3) | | | | | |
| Comparative | 3.7 | 47 | 74 | 3.5 | −33.0 |
| Example 2 | | | | | |
| (PAN/N3) | | | | | |
| Example 1 | 4.5 | 56 | 80 | 5.0 | — |
| (PSF/N5) | | | | | |
| Example 2 | 7.0 | 29 | 67 | 10.0 | −20.0 |
| (PSF/N6) | | | | | |
| Example 3 | — | — | — | 30.0 | −12.0 |
| (PAN/N5) | | | | | |
| Example 4 | 18 | 15 | 68 | 10.0 | −20.0 |
| (PAN/N6/0.1- | | | | | |
| PEI) | | | | | |
| Example 5 | 6 | 22 | 72 | 40 | −2.0 |
| (PAN/N6/1.0- | | | | | |
| PEI) | | | | | |
| Example 6 | — | — | — | 30 | −12.0 |
| (PAN/N3/1.0- | | | | | |
| PEI) | | | | | |

As presented in Table 2, it was confirmed that the nano-filtration membranes of Examples 1 to 6 had zeta potential values of 5.0 mV or higher under a pH 3 condition at which an ordinary lactic acid fermented liquor was maintained. Further, as the concentration of the PEI solution used in the formation of the PEI layer was increased, charge density was increased so that the zeta potential values of the nano-filtration membranes were increased. The nano-filtration membranes of Examples 1 to 6 exhibited substantially reduced lactic acid rejection ratios compared to nano-filtration membranes of Comparative Examples 1 and 2 having zeta potential values of lower than 5.0 mV. In contrast, the nano-filtration membranes of Examples 1 to 6 exhibited almost equal levels of glucose rejection ratios compared to the nano-filtration membranes of Comparative Examples 1 and 2 having zeta potential values of lower than 5.0 mV. Ordinarily, since lactic acid has a carboxyl group, lactic acid has negative electric charges or neutral electric charges in the fermented liquor. It is believed that lactic acid rejection ratios of nano-filtration membranes having positive-chargeability are decreased by electrical attraction between lactic acid ions and surfaces of the nano-filtration membranes having positive-chargeability. Particularly, when a polymer electrolyte such as PEI is grafted, charge density values of the nano-filtration membranes are relatively high, and selective lactic acid-recovering effects caused by electrical attraction may be further increased accordingly.

As described above, according to one or more of the above embodiments of the present invention, the following effects may be obtained. Organic acids in an organic acid-containing aqueous solution are presented in the form of “free organic acids, i.e., organic acids that are not dissociated”, or “organic acid ions”. The organic acid ions are anions. The positively chargeable polymer layer strongly attracts anionic organic acid ions based on electrical attraction. Accordingly, the positively chargeable polymer layer may strongly promote that the anionic organic acid ions permeate the positively chargeable polymer layer and the supporting membrane.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A nano-filtration membrane having a positively chargeable surface, wherein the positively chargeable surface has a surface zeta potential of about 5 mV or higher under a pH condition from about 2.0 to about 6.0.

2. The nano-filtration membrane of claim 1, wherein the positively chargeable surface comprises:
a supporting membrane; and
a positively chargeable polymer layer adhered to at least one side of the supporting membrane,
wherein the positively chargeable polymer layer has a surface zeta potential of about 5 mV or higher under a pH condition from about 2.0 to about 6.0.

3. The nano-filtration membrane of claim 2, wherein the positively chargeable polymer layer comprises a polyamide comprising an acyl chloride monomer and an amine monomer having 4 to 8-NH or —NH₂ groups.

4. The nano-filtration membrane of claim 3, wherein the positively chargeable polymer layer comprises a polyamide comprising an acyl chloride monomer and an amine monomer having 5 to 7-NH or —NH₂ groups.

5. The nano-filtration membrane of claim 3, wherein the positively chargeable polymer layer has a molecular weight cutoff of from about 100 daltons to about 1,000 daltons.

6. The nano-filtration membrane of claim 3, wherein the amine monomer comprises triethylenetetraamine (H(NHCH₂CH₂)₃NH₂), tetraethylenepentaamine (H(NHCH₂CH₂)₄NH₂), pentaethylenehexaamine (H(NHCH₂CH₂)₅NH₂), hexaethyleneheptaamine (H(NHCH₂CH₂)₆NH₂), heptaethyleneoctaamine (H(NHCH₂CH₂)₇NH₂), or a combination thereof.

7. The nano-filtration membrane of claim 3, wherein the acyl chloride monomer is 1,3,5-benzenetricarbonyl trichloride.

8. The nano-filtration membrane of claim 3, wherein a molar ratio of the amine monomer to the acyl chloride monomer is from about 5:1 to about 10:1.

9. The nano-filtration membrane of claim 3, wherein the positively chargeable polymer layer is formed by supplying an aqueous solution of the amine monomer to at least one side of the supporting membrane, and supplying an organic solution of the acyl chloride monomer to the side of the supporting membrane to which the aqueous solution of the amine monomer is supplied.

10. The nano-filtration membrane of claim 2, wherein the positively chargeable polymer layer comprises a cationic polymer electrolyte.

11. The nano-filtration membrane of claim 10, wherein the cationic polymer electrolyte comprises polyethyleneimine (PEI), poly(allylamine hydrochloride) (PAH), poly(diallyldimethylammonium chloride) (PDADMAC), or a combination thereof.

12. The nano-filtration membrane of claim 10, wherein the positively chargeable polymer layer is formed by applying a solution of the cationic polymer electrolyte to at least one side of the supporting membrane, and solidifying the applied solution of the cationic polymer electrolyte.

13. The nano-filtration membrane of claim 10, wherein the cationic polymer electrolyte comprises an —NH or —NH₂ group, and the supporting membrane comprises a polyamide comprising an amine monomer and an acyl chloride monomer.

14. The nano-filtration membrane of claim 2, wherein the positively chargeable polymer layer comprises:
an N₄₋₈ polyamide layer comprising an N₄₋₈ amine monomer and an acyl chloride monomer on the supporting membrane; and
a cationic polymer electrolyte layer on the N₄₋₈ polyamide layer.

15. The nano-filtration membrane of claim 2, wherein the positively chargeable polymer layer comprises:
an N₄₋₈ polyamide layer comprising an N₄₋₈ amine monomer and an acyl chloride monomer on the supporting membrane; and
a cationic polymer electrolyte layer having amine groups on the N₄₋₈ polyamide layer.

16. A method of separating an organic acid from a nano-filtration feed, the method comprising filtering a nano-filtration feed containing an organic acid through the nano-filtration membrane of claim 1, thus providing a nano-filtration permeate comprising the organic acid.

17. The method of claim 16, wherein the nano-filtration feed containing an organic acid is an ultra-filtration (UF) permeate obtained by filtering a culture solution through a UF membrane, the culture solution being produced through an organic acid fermentation process using microorganisms.

18. The method of claim 16, wherein the nano-filtration feed containing an organic acid has a pH value from about 2 to about 6.

19. The method of claim 16, wherein the nano-filtration feed containing an organic acid has a pH value higher than a pKa value of the organic acid.

20. The method of claim 16, further comprising: treating the nano-filtration permeate with an ion exchange resin; concentrating the nano-filtration permeate; distilling the nano-filtration permeate; or a combination thereof.

21. The method of claim 16, wherein the organic acid is lactic acid.

22. The method of claim 16, wherein the saccharide permeability is 35% or lower and the organic acid permeability is 70% or higher.
